(19) United States
(12) Patent Application Publication
(10) Pub. No.: US 2013/0210923 A1
(43) Pub. Date: Aug. 15, 2013

(54) Antimicrobial Hand Soap Composition

(71) Applicant: Yun-Peng Zhu, Fairlawn, NJ (US)

(72) Inventor: Yun-Peng Zhu, Fairlawn, NJ (US)

(73) Assignee: Reckitt & Colman (Overseas) Limited, Berkshire (GB)

(21) Appl. No.: 13/520,903

(22) PCT Filed: Dec. 14, 2010
(86) PCT No.: PCT/GB10/52084

§ 371 Date: Apr. 29, 2013

(30) Foreign Application Priority Data
Jan. 6, 2010 (GB) 1000122.0

(51) Int. Cl. A61K 8/41; A61Q 19/10

(52) U.S. Cl. CPC A61K 8/416; A61Q 19/10
USPC 514/643

(57) ABSTRACT

Topical aqueous liquid compositions for application to the epidermis which provide a germicidal benefit thereto comprising: 0.02-10% wt. of a germicidally effective amount of a quaternary ammonium surfactant compound, 0.1-25% wt. of at least one anionic surfactant constituent which excludes anionic surfactants of the sulfate and sulfonate types, 0.1-15% wt. of a cationic conditioner constituent, 0-10% wt. of a foam stabilizer constituent; at least 70% wt. of water, and optionally one or more of optional constituents which may be used to impart an aesthetic benefit, e.g., color, fragrance, or a further technical benefit, e.g., viscosity adjustment, to the liquid compositions, characterized in that: (a) the weight ratio of the germicidally effective quaternary ammonium and cationic conditioner constituent to the anionic surfactant constituent is not less than 0.6; and/or, (b) the weight ratio of the germicidally effective quaternary ammonium surfactant compound to the cationic conditioner constituent is less than 0.14; and/or, (c) the ratio of the cationic conditioner constituent to the anionic surfactant constituent is at least 0.5; and, (d) the pH of the liquid composition is between about 3.5 and about 7.5.

The present invention relates to a liquid composition which is particularly useful in concurrently providing a cleaning treatment and an antimicrobial benefit to surface, particularly when topically applied to a part of a body, e.g., human or animal. In certain preferred embodiments the inventive compositions are formulated as liquid hand soap compositions useful in treating dermal surface, e.g. the hands, arms, face etc.

Topical compositions, per se, are well-known in the cosmetic, dermatological as well as in the pharmaceutical fields. Most topical compositions are intended to provide at least one but generally provide multiple or more specific benefits after being applied to the human skin. For example, personal care compositions which are primarily intended to be soaps for general cleaning of the human skin such as hand soaps or body wash soaps are well known in the fields of cosmetics and personal care products. While providing a primary cleaning benefit, such personal care compositions frequently also provide ancillary benefits such as moisturizing and nourishing the skin. Such personal care compositions which provide a good general cleaning benefit are usually based on one or more anionic soaps or anionic surfactants which are recognized to provide good cleaning and good foaming. However, such compositions typically provide only limited germicidal benefits.

Also known to the art are topical compositions which are primarily directed to provide an germicidal benefit to the epidermis or other body part when applied thereto. Such typically take the form of viscous gels and are often largely comprised of an alcohol, usually ethanol, with further constituents, e.g., thickeners. While often technically effective to provide a germicidal benefit, such compositions are also not without shortcomings, including in some cases, an unpleasant skin feel and in other cases, an unpleasant odor, as well as an undesired drying effect to the skin.

The compositions disclosed in published patent application US 2003/0190302 disclose liquid compositions which include quaternary ammonium compounds but which require one or more anionic surfactants selected from alkyl sulfates or alkyl ether sulfates.

It is to these shortcomings as well as further shortcomings in the art to which the current invention is directed.

In a first aspect of the invention there are provided a liquid composition for the treatment of the skin (epidermis) as well as other body surfaces including the hair which liquid composition provides an effective cleaning and/or germicidal benefit.

According to a second aspect of the invention is provided a method for the manufacture or production of the liquid composition as set forth herein.

According to a still further aspect of the invention there is provided an improved method for the treatment of the skin (epidermis) as well as other body surface including the hair, which method includes the application of a cleaning and/or germicidally effective amount of the liquid composition described herein in order to provide an effective cleaning and/or germicidal benefit.

These and further aspects of the invention are provided as described within this specification.

Broadly stated, in a first aspect of the invention there are provided aqueous liquid compositions for application to the epidermis, e.g., hands, arms, legs, face, scalp as well as other body areas and which provide a germicidal benefit thereto. These liquid compositions comprise (in certain embodiments, consist essentially of, or consist of):

0.02-10% wt. of a germicidally effective amount of a quaternary ammonium surfactant compound, which simultaneously functions as a source of cations and also to impart a germicidal benefit to the epidermis upon which the liquid composition is applied;

0.1-25% wt. of at least one anionic surfactant constituent which excludes anionic surfactants of the sulfate and sulfonate types, which anionic surfactant constituent simultaneously provides a cleaning benefit and which also functions as a source of anions;

0.1-15% wt. of a cationic conditioner constituent, which simultaneously functions as an additional source of cations, which may provide an ancillary antimicrobial benefit, and which preferably also may impart a conditioning benefit to the epidermis upon which the liquid composition is applied;

0-10% wt. of a foam stabilizer constituent;

at least 70% wt. of water, and further

optionally one or more of optional constituents which may be used to impart an aesthetic benefit, e.g., color, fragrance, or a further technical benefit, e.g., viscosity adjustment, to the liquid compositions

characterized in that:

(a) the weight ratio of the germicidally effective quaternary ammonium and cationic conditioner constituent to the anionic surfactant constituent is not less than 0.6; and/or,

(b) the weight ratio of the germicidally effective quaternary ammonium surfactant compound to the cationic conditioner constituent is less than 0.14; and/or,

(c) the ratio of the cationic conditioner constituent to the anionic surfactant constituent is at least 0.5; and,

(d) the pH of the liquid composition is between about 3.5 and about 7.5.

In preferred embodiments the liquid compositions provide a topical germicidal composition characterized in that the said composition is effective against one or more, preferably at least two or more of the following microorganisms: E. coli, P. aeruginosa. and Vancomycin resistant Enterococcus faecalis (“VRE”).

The liquid compositions necessarily comprise a germicidally effective amount of at least one quaternary ammonium surfactant compound, which simultaneously functions as a source of cations and also to impart a germicidal benefit to the epidermis upon which the liquid composition is applied. Non-limiting examples of preferred cationic surfactant compounds and which are especially preferred are quaternary ammonium compounds and salts thereof, which may be characterized by the general structural formula:

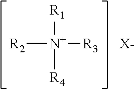

where at least one of R₁, R₂, R₃ and R₄ is a alkyl, aryl or alkylaryl substituent of from 6 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons usually containing no more than 12 carbon atoms. The substituents R₁, R₂, R₃ and R₄ may be straight-chained or may be branched, but are preferably straight-chained, and may include one or more amide, ether or ester linkages. The counterion X may be any salt-forming anion which permits water solubility or water miscibility of the quaternary ammonium complex, e.g., chloride, bromide, methosulfate, ethosulfate, lactate, saccharinate, acetate or phosphate. Preferred quaternary ammonium compounds which act as germicides according to the foregoing formula are those in which R₂ and R₃ are the same or different C₈-C₁₂alkyl, or R₂ is C₁₂₋₁₆alkyl, C₈₋₁₈alkylethoxy, C₈₋₁₈alkylphenolethoxy and R₃ is benzyl, and X is a halide, for example chloride, bromide or iodide, or is a methosulfate anion. The alkyl groups recited in R₂ and R₃ may be straight-chained or branched, but are preferably substantially linear. Mixtures of two or more different quaternary ammonium surfactant compound are expressly contemplated as being useful.

The at least one quaternary ammonium surfactant compound is necessarily present in amounts which provide or impart a germicidal benefit to dermal surfaces or other body surfaces upon which the liquid compositions taught herein are applied. Advantageously the at least one quaternary ammonium surfactant compound is present in amounts of from about 0.025-10% wt., preferably are present in amounts of from about 0.1-5% wt. of the liquid composition of which it forms a part. Particularly preferred amounts of the at least one quaternary ammonium surfactant compound in liquid compositions are disclosed in one or more of the Examples which are disclosed hereafter.

The liquid compositions of the invention also necessarily includes an anionic surfactant constituent which includes at least one anionic surfactant compound excluding anionic surfactants compounds of the sulfate and sulfonate types, which anionic surfactant constituent simultaneously provides a cleaning benefit and which also functions as a source of anions.

Anionic surfactant compounds useful in the anionic surfactant constituent can be one or more of: alcohol phosphates and phosphonates, alkyl carboxylates, alkyl ether carboxylates, alkyl alkoxy carboxylates especially those having 1 to 5 moles of ethylene oxide, octoxynol phosphates, nonoxynol phosphates, taurates, fatty taurides, alkyl phosphates, isethionates, N-acyl taurates, alkyl succinamates alkyl polyethoxy carboxylates, and sarcosinates.

Exemplary anionic surfactant compounds include alkyl ether carboxylates, particularly those having the general structural formula:

R—O—(CₙH₂ₙO)ₘ—R₁—COO⁻M⁺

wherein R is a straight or branched, long chain, alkyl group containing from 8 to 18 carbon atoms, n is an integer from 2 to 4, m is an integer from 1 to 100, R₁ is CH₂, CH₂CH₂, or CH₂CH₂CH₂, and M is a counterion such as an organic or inorganic cation including singly valent cations as well as polyvalent cations. Exemplary cations include cations of an alkali metal including sodium or lithium, or organic cations such as ammonium, diethylammonium, or triethylammonium cations, as well as other cations not particularly recited here. Such anionic alkyl ether carboxylates are known to be useful as surfactant compositions, and find use in the liquid compositions taught herein. Such surfactants are presently commercially available under the trade name SANDOPAN (ex. Clariant Chemical Corp.), NEODOX 25-6 and NEODOX 23-4 (ex. Shell Chemical Co.), SURFINE WLG (ex. Finetex Inc.), as well as MIRANATE LEC and MIRANATE LEC-80 (ex. Rhodia) both generally described to be sodium laureth-13 carboxylate surfactants, of which MIRANATE LEC-80 is particularly preferred, and is described as comprising a C₁₂ straight chained alkyl group, 13 ethoxy groups and a sodium cation. As supplied, MIRANATE LEC-80 includes about 79% active ingredient supplied in an aqueous/alcoholic carrier.

Exemplary anionic surfactant compounds also include sarcosinate surfactants which are alkali metal salts of N-alkyl-N-acyl amino acids. These are salts derived from the reaction of (1) N-alkyl substituted amino acids of the formula:

R₁—NH—CH₂—COOH

where R₁ is a linear or branched chain lower alkyl of from 1 to 4 carbon atoms, especially a methyl, for example, aminoacetic acids such as N-methylaminoacetic acid (i.e. N-methyl glycine or sarcosine), N-ethyl-aminoacetic acid, N-butylaminoacetic acid, etc., with (2) saturated natural or synthetic fatty acids having from 8 to 18 carbon atoms, especially from 10 to 14 carbon atoms, e.g. lauric acid, and the like.

The resultant reaction products are salts which may have the formula:

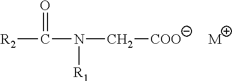

where M is an alkali metal ion such as sodium, potassium or lithium; R₁ is as defined above; and wherein R₂ represents a hydrocarbon chain, preferably a saturated hydrocarbon chain, having from 7 to 17 carbon atoms, especially 9 to 13 carbon atoms of the fatty acyl group

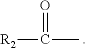

Exemplary useful sarcosinate surfactants include cocoyl sarcosinate, lauroyl sarcosinate, myristoyl sarcosinate, palmitoyl sarcosinate, stearoyl sarcosinate and oleoyl sarcosinate, tallow sarcosinate, myristoyl/stearoyl sarcosinates, as well as salts thereof such as sodium lauroyl sarcosinate, sodium cocoyl sarcosinate, and sodium myristoyl sarcosinates. Such sarcosinates are presently commercially available from Croda Inc., and are marketed as CRODASINIC surfactants.

Advantageously the anionic surfactant constituent is present in amounts of from about 0.1-25% wt., preferably are present in amounts of from about 0.5-10% wt. of the liquid composition of which it forms a part. Particularly preferred amounts of the anionic surfactant constituent in liquid compositions of the invention are disclosed in one or more of the Examples which are disclosed hereafter.

In preferred embodiment, the weight ratio of the germicidally effective quaternary ammonium and cationic conditioner constituent to the anionic surfactant constituent is not less than 0.6, but preferably is at least 1, still more preferably is at least 1.5, and especially preferably is at least 3.

The inventive compositions also include a cationic conditioner constituent, which simultaneously functions as an additional source of cations, which may provide an ancillary antimicrobial benefit, and which preferably may also impart a conditioning benefit to the epidermis or other body surfaces upon which the liquid composition is applied. The cationic conditioner constituent may be one or more Polyquarternium-type polymers. Such materials, are, per se, well known to the art of topical compositions. Various grades of such cationic polymers may be used, inter alia: Polyquarternium 1; Polyquarternium 2; copolymers of hydroxyethylcellulose and diallyldimethyl ammonium chloride commercially available as Polyquarternium 4; homopolymers of diallyldimethylammonium chloride commercially available as Polyquarternium 5; dimethyldiallylammonium chloride homopolymer commercially available as Polyquarternium 6; copolymers of diallyldimethylammonium chloride with acrylamide commercially available as Polyquarternium 7; the polymeric quaternary ammonium salt of methyl and steardyl dimethylaminoethyl methacrylate quaternized with dimethyl sulfate commercially available as Polyquarternium 8; the polymeric quaternary ammonium salt of polydimethylaminoethyl methacrylate quaternized with methyl bromide commercially available as Polyquarternium 9; a polymeric quaternary ammonium salt formed from the reaction of hydroxyethyl cellulose with a trimethylammonium substituted epoxide commerically available as Polyquarternium 10; a polymeric quaternary ammonium polymer formed by the reaction of vinyl pyrrolidine and dimethyl aminoethylmethacrylate commercially available as Polyquarternium 11; a polymeric quaternary ammonium salt prepared by the reaction of ethyl methacrylate/abietyl methacrylate/diethylaminoethyl methacrylate copolymer with dimethyl sulfate commercially available as Polyquarternium 12; a polymeric ammonium salt prepared by the reaction of ethyl methacrylate/oleyl methacrylate/diethylaminoethyl methacrylate copolymer with dimethyl sulfate commercially available as Polyquarternium 12; a polymeric quaternary ammonium salt prepared by the reaction of ethyl methacrylate/oleyl methacryalte/diethylaminoethyl methacrylate copolymer with dimethyl sulfate commercially available as Polyquarternium 13; Polyquarternium 14; the copolymer of methacrylamide and betamethacrylyloxyethyl trimethyl ammonium chloride commercially available as Polyquarternium 15; the polymeric quaternary ammonium salt formed from methylvinylimidazolium chloride and vinylpyrrolidone commercially available as Polyquarternium 16; polymeric quaternary salts prepared by the reaction of adipic acid and dimethylaminopropylamine reached with dichloroethyl ether commercially available as Polyquarternium 17; a polymeric quaternary salt prepared by the reaction of azelaic acid and dimethylaminopropylamine reacted with dichloroethyl ether commercially available as Polyquarternium 18; a polymeric quaternary ammonium salt prepared by the reaction of polyvinyl alcohol with 2,3-epoxy-propylamine commercially available as Polyquarternium 19; a polymeric quaternary ammonium salt prepared by the reaction of polyvinyl octadecyl ether with 2,3-epoxypropylamine commercially available as Polyquarternium 20; copolymers of acrylic acid and dimethyldiallylammonium chloride commercially available as Polyquarternium 22; polymeric quaternary ammonium salts of hydroxyethyl cellulose reacted with lauryl dimethyl ammonium-substituted epoxide commercially available as Polyquarternium 24; a block copolymer formed by the reaction of Polyquarternium 2 and Polyquarternium 17 commercially available as Polyquarternium 27; a polymeric quaternary ammonium salt consisting of vinylpyrrolidone and dimethylaminopropyl methacrylamide monomers commercially available as Polyquarternium 28; chitosans reacted with propylene oxide and quaternized with epichlorohydrin commercially available as Polyquarternium 29; Polyquarternium 30; a polymeric quaternary ammonium salt prepared by the reaction of DMAPA acrylates/acrylic acid/acrylonitrogens copolymer with diethyl sulfate commercially available as Polyquarternium 31; Polyquarternium 32; Polyquarternium 33; Polyquarternium 34; Polyquarternium 35; Polyquarternium 36; Polyquarternium 37; polymeric quaternary ammonium salts of the terpolymer of acrylic acid/diallyldimethylammonium chloride/acrylamide commercially available as Polyquarternium 39; Polyquarternium 42; a copolymer of acrylamide, acrylamidopropyltrimonium chloride, 2-aminopropylacrylamide sulfonate and DMAPA polymers commercially available as Polyquarternium 43; a polymeric quaternary ammonium salt consisting of vinylpyrrolidone and quaternized imidazoline monomers commercially available as Polyquarternium 44; Polyquarternium 45; a polymeric quaternary ammonium salt prepared by the reaction of vinylcaprolactam and vinylpyrrolidone with methylvinylimidazolium commercially available as Polyquarternium 46; a polymer quaternary ammonium chloride formed by the polymerization of acrylic acid with methacrylamidopropyl trimethylammonium chloride and methylacrylate commercially available as Polyquarternium 47; a copolymer of methacryloyl ethyl betaine, 2-hydroxyethyl methacrylate and metacyloyl ethyl trimethyl ammonium chloride commercially available as Polyquarternium 48; a copolymer of methacryloyl ethyl betaine, PEG-9 methacrylate and methacryloyl ethyl trimethyl ammonium chloride commercially available as Polyquarternium 49; Polyquarternium 50; Polyquarternium 51; Polyquarternium 52; a copolymer of acrylic acid, acrylamide and methacrylamidopropyltrimonium chloride commercially available as Polyquarternium 53; a polymeric quaternary ammonium salt prepared by the reaction of aspartic acid and C6-C18 alkylamine with dimethylaminopropylamine and sodium chloroacetate commercially available as Polyquarternium 54; a polymeric quaternary ammonium chloride formed by the reaction of vinylpyrrolidone, dimethylaminopropyl methacrylamide and methacryloylaminopropyl lauryldimonium chloride commercially available as Polyquarternium 55; and a polymeric quaternary ammonium salt consisting of isophorone diisocyanate, butylene glycol and dihydroxyethyldimonium methosulfate monomers commercially available as Polyquarternium 56. Each of the foregoing are described in the literature, particularly in the International Cosmetic Ingredient Dictionary and Handbook, Volume 2 (9^{th} Edition, 2002), at pages 1311-1319. Other polyquarternium compounds although not specifically elucidated here may also be utilized in the present inventive compositions.

The cationic conditioner constituent may also be one or more monoalkyl amine derivatives of quaternary ammonium compounds, e.g.; cetyl trimethyl ammonium bromide, cetyl trimethyl ammonium chloride, myristyl trimethyl ammonium bromide, (also referred to as Quaternium-13 4), stearyl dimethyl benzyl ammonium chloride, (also known as stearalkonium chloride 5), oleyl dimethyl benzyl ammonium chloride, (also known as olealkonium chloride 6), lauryl/myristryl trimethyl ammonium methosulfate, (also known as cocotrimonium methosulfate 7), cetyl-dimethyl-(2)hydroxyethyl ammonium dihydrogen phosphate, (also known as hydroxyethyl cetyldimonium phosphate 8), and the like.

The cationic conditioner constituent may also be one or more dialkyl amine derivatives of quaternary ammonium compounds, e.g.; distearyldimonium chloride, dihydrogenated palmoylethyl hydroxyethylmonium methosulfate, dipalmitoylethyl hydroxyethylmonium methosulfate, dioleoylethyl hydroxyethylmonium methosulfate, and hydroxypropyl bisstearyldimonium chloride, and the like.

The cationic conditioner constituent may also be one or more imidazoline derivatives, e.g.; isostearyl benzylimidonium chloride, cocoyl benzyl hydroxyethyl imidazolinium chloride, cocoyl hydroxyethylimidazolinium PG-chloride phosphate, stearyl hydroxyethylimidonium chloride, and the like.

The cationic conditioner constituent may also be one or more of: babassuamidopropylkonium chloride, cocotrimonium chloride, distearyldimonium chloride, wheat germ-amidopropalkonium chloride, stearyl octyldimonium methosulfate, isostearaminopropal-konium chloride, dihydroxypropyl PEG-5 linoleaminium chloride, PEG-2 stearmonium chloride, behentrimonium chloride, dicetyl dimonium chloride, behentrimonium methosulfate, tallow trimonium chloride and behenamidopropyl ethyl dimonium ethosulfate, and the like.

The cationic conditioner constituent is advantageously present in the liquid compositions of the invention in amounts of from about from 0.1-15% wt., preferably in amounts from 2-12% wt., based on the total weight of liquid composition of which they form a part.

In preferred embodiments the weight ratio of the germicidally effective quaternary ammonium surfactant compound to the cationic conditioner constituent is less than 0.14 by weight.

In preferred embodiments the weight ratio of the cationic conditioner constituent to the anionic surfactant constituent is at least 0.5; preferably is at least 1 and especially preferably is at least 2.

Optionally but very preferably the liquid compositions also include a foam stabilizer constituent. Such a foam stabilizer constituent is advantageously one or more nonionic surfactants but is preferably an amphoteric or zwitterionic surfactant, or a fatty alcoholamide.

Exemplary useful foam stabilizers are amine oxide surfactants, which may be:

alkyl di(C₁-C₇) amine oxides in which the alkyl group has about 10-20, and preferably 12-16 carbon atoms, and can be straight or branched chain, saturated or unsaturated. Examples of such compounds include lauryl dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide;

alkyl di(hydroxy C₁-C₇) amine oxides in which the alkyl group has about 10-20, and preferably 12-16 carbon atoms, and can be straight or branched chain, saturated or unsaturated. Examples of such compounds include bis(2-hydroxyethyl) cocoamine oxide, bis(2-hydroxyethyl) tallowamine oxide; and bis(2-hydroxyethyl) stearylamine oxide;

alkylamidopropyl di(C₁-C₇) amine oxides in which the alkyl group has about 10-20, and preferably 12-16 carbon atoms, and can be straight or branched chain, saturated or unsaturated. Examples of such compounds include cocoamidopropyl dimethyl amine oxide and tallowamidopropyl dimethyl amine oxide; and

alkylmorpholine oxides in which the alkyl group has about 10-20, and preferably 12-16 carbon atoms, and can be straight or branched chain, saturated or unsaturated. Particularly preferred are alkyl di(C₁-C₂) amine oxides in which the alkyl group has about 10-14, and preferably has 12 carbon atoms, which are preferably saturated. Especially preferred is lauryl dimethyl amine oxide which in preferred embodiments is present to the exclusion of other amine oxides.

Further exemplary foam stabilizers are water dispersible and water soluble betaine surfactants. Exemplary betaine surfactants include those which may be represented by the general formula:

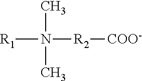

wherein R₁ is an alkyl group containing from 8 to 18 carbon atoms, or the amido radical which may be represented by the following general formula:

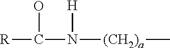

wherein R is an alkyl group having from 8 to 18 carbon atoms, a is an integer having a value of from 1 to 4 inclusive, and R2 is a C1-C4 alkylene group. Examples of such water-soluble betaine surfactants include dodecyl dimethyl betaine, as well as cocoamidopropylbetaine.

One or more amphoacetates such as sodium lauroamphoacetate, or diamphoacetates may also be used. Amphoacetates may be represented by the following general formula:

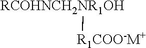

and, diamphoacetates may be represented by the following general formula:

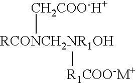

wherein in both formulas, R represents an aliphatic group having 8 to 18 carbon atoms, R₁ represents an aliphatic group having 1 to 5 carbon atoms, but is preferably —CH₂—, or —CH₂CH₂—, and M is a cation such as sodium, potassium, ammonium, or a substituted ammonium. Examples of such compounds include: sodium lauroamphoacetate, sodium cocoamphoacetate, disodium lauroamphoacetate, and disodium cocoamphoacetate.

The foam stabilizer constituent may also comprise one or more amphoteric surfactants. Exemplary useful amphoteric surfactants include those which may be represented by the following general formula

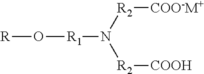

in which, R represents a C₄ to C₂₄ alkyl group, and is preferably a C10 to C16 alkyl group, R1 and R2 independently represent a C₁ to C₈ alkyl group, is preferably —CH₂CH₂— or —CH₂CH₂CH₂—, and M may be any salt-forming anion which permits water solubility or water miscibility of the compound, e.g., chloride, bromide, methosulfate, ethosulfate, lactate, saccharinate, acetate or phosphate. Such compounds are presently commercially available, such as those marketed in the Tomamine Amphoteric series of amphoteric surfactants, ex. Air Products Inc.

The foam stabilizer constituent may also comprise at least one a fatty alkanolamide examples of which include but are not limited to: cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof.

In addition to a foam boosting benefit, the inclusion of a fatty alkanolamide may provide an ancillary thickening benefit as well.

When present, the foam stabilizer constituent is advantageously present in amounts of up to about 10% wt., based on the total weight of the liquid composition of which it forms a part. Preferably a foam stabilizer constituent is present in an amount of between about 0.1-7.5% wt., and especially preferably between about 0.5-5% wt. In particularly preferred embodiments a foam stabilizer constituent is necessarily present in the liquid compositions of the invention.

The liquid compositions of the present invention are largely aqueous in nature, and advantageously comprise at least about 50% wt., and in order of increasing preference at least about: 55% wt., 60% wt., 65% wt., 70% wt., 75% wt., 80% wt., 85% wt., 90% wt. of water. The water may be tap water, but is preferably distilled and is most preferably deionized water.

The liquid compositions of the invention exhibit a pH in the range of from about to about 3.5 and about 7.5, but preferably exhibit a pH of between about 4 to about 6.6. Especially preferred pH ranges and pH values are disclosed in one or more of the Examples disclosed hereinafter.

The liquid compositions of the invention may include one or more further optional constituents which may be used to improve one or more aesthetic and/or provide one or more technical characteristics of the liquid composition of which they form a part. Typically they are included in only small amounts, and usually the total amount of any such optional constituents docs not exceed 50% wt. of the liquid compositions of which they form a part.

Optionally, the liquid compositions of the invention may include one or more nonaqueous solvents, e.g., organic solvents. By way of non-limiting example exemplary useful organic solvents which may be included in the liquid compositions include those which are at least partially water-miscible such as alcohols (e.g., low molecular weight alcohols, such as, for example, ethanol, propanol, isopropanol, and the like), glycols (such as, for example, ethylene glycol, propylene glycol, hexylene glycol, and the like), water-miscible ethers (e.g. diethylene glycol diethylether, diethylene glycol dimethylether, propylene glycol dimethylether), water-miscible glycol ether (e.g. propylene glycol monomethylether, propylene glycol mono ethylether, propylene glycol monopropylether, propylene glycol monobutylether, ethylene glycol monobutylether, dipropylene glycol monomethylether, diethyleneglycol monobutylether), lower esters of monoalkylethers of ethylene glycol or propylene glycol (e.g. propylene glycol monomethyl ether acetate), and mixtures thereof. Glycol ethers having the general structure Rₐ—R_{b}—OH, wherein Rₐ is an alkoxy of 1 to 20 carbon atoms, or aryloxy of at least 6 carbon atoms, and R_{b} is an ether condensate of propylene glycol and/or ethylene glycol having from one to ten glycol monomer units. Of course, mixtures of two or more organic solvents may be used concurrently. When present the one or more nonaqueous solvents may comprise to about 25% wt., preferably not more than about 20% wt. of the liquid compositions of which they form a part.

The liquid compositions of the invention may optionally comprise one or more emollients which provide softness to surfaces treated with the liquid compositions.

The liquid compositions may include skin conditioning agents. Such include cationic Polyquarternium-type polymers which are known to the art of topical compositions. Various grades of such cationic polymers may be used, inter alia: homopolymers of diallyldimethylammonium chloride commercially available as Polyquarternium 5; dimethyldiallylammonium chloride homopolymer commercially available as Polyquarternium 6; copolymers of diallyldimethylammonium chloride with acrylamide commercially available as Polyquarternium 7; polymeric quaternary ammonium salt derived from the reaction of hydroxyethyl cellulose with a trimethylammonium substituted epoxide commercially available as polymeric quaternary ammonium salts derived from the reaction of hydroxyethyl cellulose with a trimethylammonium substituted epoxide commercially available as Polyquarternium 10; copolymers of 1-vinyl-2-pyrrolidine and dimethylaminoethyl methacrylate commercially available as Polyquarternium 11; Polyquarternium 15; copolymers of 1-vinyl-2-pyrrolidine and 1-vinyl-3-methyl-imidazolium salt commercially available as Polyquarternium 16; copolymers of acrylic acid and dimethyldiallylammonium chloride commercially available as Polyquarternium 22; polymeric quaternary ammonium salts of hydroxyethyl cellulose reacted with lauryl dimethyl ammonium-substituted epoxide commercially available as Polyquarternium 24; Polyquarternium 28; polymeric quaternary ammonium salts of the terpolymer of acrylic acid/diallyldimethylammonium chloride/acrylamide commercially available as Polyquarternium 39; Polyquarternium 44; Polyquarternium 46; and, terpolymers of acrylic acid with methacrylamidopropyl trimethylammonium chloride and methylacrylate commercially available as Polyquarternium 47. Other polyquarternium compounds although not specifically elucidated here may also be utilized in the present inventive compositions. It is noted that one or more such Polyquarternium type polymers disclosed here with reference to their utility as skin conditioning agents may simultaneously function as the cationic conditioner constituent discussed previously, and may be concurrently used for that purpose as well. When included in the inventive compositions, the one or more cationic polyquarternium-type polymers or other known art skin conditioning agents may advantageously be present in amounts of from about from 0.001-15% wt., based on the total weight of the liquid composition of which they form a part.

Further optional constituents which may be included in the liquid compositions include emollients such as one or more of esters, fatty acids and alcohols, polyols and hydrocarbons which may impart a softening effect when topically applied. Exemplary esters include mono- and di-esters which may be, inter alia, dibutyl adipate, diethyl sebacate, diisopropyl dimerate, dioctyl succinate, Exemplary branched chain fatty esters include 2-ethyl-hexyl myristate, isopropyl stearate and isostearyl palmitate. Exemplary tribasic acid esters include triisopropyl trilinoleate and trilauryl citrate. Exemplary straight chain fatty esters include lauryl palmitate, myristyl lactate, oleyl eurcate and stearyl oleate. Further exemplary useful esters include coco-caprylate/caprate (a blend of coco-caprylate and coco-caprate), propylene glycol myristyl ether acetate, diisopropyl adipate and cetyl octanoate. Exemplary useful fatty alcohols and acids include, inter alia, those compounds having from 10 to 20 carbon atoms, preferentially cetyl, myristyl, palmitic and stearyl alcohols and acids.

The emollient constituent may be a single compound or a mixture of two or more compounds which provide a beneficial emollient effect. When present in the liquid compositions of the invention. the total amount of the emollient constituent(s) present are sufficient to provide an improved softening effect to the liquid compositions and are advantageously included in amounts of from 0.05-10% wt. based on the total weight of the topical germicidal compositions of which they form a part. When present, the emollient constituent(s) are preferably present in amounts from 0.05-10% wt., but are most desirably present in reduced weight percentages from about 0.1-5% wt. based on the total weight of the liquid composition of which they form a part.

The liquid compositions of the invention may also include one or more dimethicones or silicones as further skin treatment or skin protectant constituents, which, when present, may be included in effective amounts. When present such one or more further skin treatment or skin protectant constituents are advantageously present in amounts from 0.01-10% wt., but are most desirably present in reduced weight percentages from about 0.1-5% wt. based on the total weight of the liquid compositions of which they form a part.

The liquid compositions may optionally comprise one or more humectants, including polyhydric alcohols including polyalkylene glycols as well as alkylene polyols and their derivatives, inter alia, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. Still further humectants include polyols e.g., linear and branched chain alkyl polyhydroxyl compounds such as, propylene glycol, polyethylene glycol, glycerine and sorbitol. Exemplary hydrocarbons which may also serve as humectants are those having hydrocarbon chains anywhere from 12 to 30 carbon atoms, particularly, mineral oil, petroleum jelly, squalene and isoparaffins.

The humectants may be used singly or two or more humectants may be included in the liquid compositions of the invention. When present, in accordance with certain of the preferred embodiments, one or more humectants may be included in effective amounts, advantageously from 0.01-25% wt., preferably from 5-15% wt. based on the total weight of the liquid composition of which it forms a part. In particularly preferred embodiments, a humectant is necessarily present in an amount of from 5-12.5% wt., and a particularly preferred humectant is selected from polyhydroxy alcohols, such as glycerine, and/or alkoxlated polyhydroxy alcohols, such as ethoxylated glycerine and propoxylated glycerine.

The liquid compositions may include one or more powders or pulvurent materials. These powders include mica, chalk, talc, Fullers earth, kaolin, starch, silica, silicates, hydrated aluminum silicate, fumed silica, aluminum starch octenyl succinate as well as comminuted or particulate polymers such as particles of polyamides (Nylons), polyalkyleneterephtalates (PET, PBT), polyolefins (PE) or fluoropolymers (polytetrafluoroethylene) as well as mixtures of two or more thereof. The inclusion of one or more powders in the inventive liquid compositions may provide an improved tactile benefit and/or may provide an opacifying effect to the compositions. Preferred powders are those based on inorganic materials, e.g., silica, silicates and talc. Such are typically provided to the liquid compositions as finely divided particles. While such powders may be included in any effective amount, when present they are advantageously included in amounts of between about 0.01% wt. to about 5% wt., preferably between about 0.25% wt. to about 2% wt., based on the total weight of the liquid composition of which they form a part.

Optionally, but in some instances preferably, the liquid compositions of the invention include a preservative constituent. Exemplary preservatives may be added in minor amounts and include those which are presently commercially available, e.g., under the trademarks Kathon® CG/ICP (Rohm & Haas, Philadelphia Pa.), Suttocide® A (Sutton Labs, Chatham N.J.) as well as Midtect® TFP (Tri-K Co., Emerson, N.J.). Such may be included in minor amounts, e.g., 0.0001 to about 0.5% by weight of the total concentrate composition, more generally an amount of about 0.2% by weight and less.

The liquid compositions may include a fragrance constituent, which may be based on natural and synthetic fragrances and most commonly are mixtures or blends of a plurality of such fragrances, optionally in conjunction with a carrier such as an organic solvent or a mixture of organic solvents in which the fragrances are dissolved, suspended or dispersed. When present in a liquid composition, the fragrance constituent may be present in any effective amount such that it can be discerned by a consumer of the said composition, however is advantageously present in amounts of up to about 5% wt., preferably from about 0.00001% wt. to about 1.5% wt., most preferably from about 0.0001% wt. to 0.25% wt. based on the total weight of the liquid composition of which it forms a part.

The inventive liquid compositions may include one or more colorants, e.g, dyes or pigments which are known to the art be useful in cosmetic or topical compositions which may be used to impart a desired color or tint to the inventive compositions. Exemplary colorants include pigments, inter alia, inorganic red pigments, such as iron oxide, iron hydroxide and iron titanate; inorganic brown pigments, such as gamma-iron oxide; inorganic yellow pigments, such as iron oxide yellow and loess; inorganic black pigments, such as iron oxide black and carbon black; inorganic violet pigments, such as manganese violet and cobalt violet; inorganic green pigments, such as chromium hydroxide, chromium oxide, cobalt oxide and cobalt titanate; inorganic blue pigments, such as Prussian blue and ultramarine blue; lakes of tar pigments; lakes of natural dyes; and synthetic resin powder complexes of the inorganic pigments as recited above. Advantageously one or more colorants may be added in amounts of about 0.001% wt. to about 0.1% by weight, based on the total weight of the liquid composition of which the colorant(s) forms a part.

The liquid compositions of the invention may one or more essential oils which are selected to provide a so-called “aromatherapy benefit” or “holistic benefit” to the user. Essential oils are complex mixtures of different organic molecules, such as terpenes, alcohols, esters, aldehydes, ketones and phenols. Such essential oils are frequently extracted from naturally occurring botanical sources such as flowers, stems, leaves, roots and barks of aromatic plants. While essential oils may be used singly, it is also common to utilize blends of essential oils in order to provide a conjunctive aroma benefit, aromatherapy benefit, holistic benefit and possibly a therapeutic benefit as well.

Preferred essential oils providing an aromatherapy benefit for use in the liquid compositions of the present invention include one or more selected from chamomile oil, lavendin oil, lavender oil, grapefruit oil, lemon oil, line oil, mandarin orange oil, orange flower oil and orange oil. Chamomile oil may be used to promote both a fresh, clean and attractive scent and possibly provide a stress-relaxing benefit to the user of the inventive composition. Lavender oil, and lavendin, may be used to promote both a fresh and attractive scent and possibly also provide a stress-relaxing benefit to the user of the inventive liquid composition. One or more of grapefruit oil, lemon oil, line oil, mandarin orange oil, orange flower oil and orange oil provide a clean citrus scent and may possibly impart a perceived therapeutic benefit as well when used.

As used in the present invention, these one or more essential oils providing an aromatherapy benefit or holistic benefit are present in an amount about 0.00001 wt. % to about 1 wt. %, preferably from about 0.00005 wt. % to about 0.75 wt. %, and more preferably from about 0.0001 wt. % to about 0.5 wt. % of the total weight of the liquid composition. It is to be understood that these one or more essential oils providing an aromatherapy benefit may be used with our without the optional fragrancing constituent recited previously and may be used wholly or partially in place of said fragrancing constituent.

The liquid compositions may include one or more antioxidant constituents; certain of these antioxidant constituents may additionally provide an anti-wrinkling benefit to the skin or other topical treatment benefit. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide), as well as oil-soluble antioxidants such as butylated hydroxytoluene, retinoids, tocopherols e.g., tocopherol acetate, tocotrienols, and ubiquinone, natural extracts containing antioxidants such as extracts containing flavonoids and isoflavonoids and their derivatives, extracts containing resveratrol and the like, as well as certain natural extracts e.g., grape seed, green tea, pine bark, propolis, and the like. When present the total amount of such antioxidants are usually not in excess of 5% wt, preferably from 0.0001-4% wt. based on the total weight of the liquid compositions of which it forms a part.

Optionally the liquid compositions may include one or more vitamins. Examples of vitamins which can be added include vitamin A, such as vitamin A oil, retinol, retinyl acetate and retinyl palmitate; vitamin B, including vitamin B₂ such as riboflavin, riboflavin butyrate and flavin adenine nucleotide, vitamin B₆ such as pyridoxine hydrochloride, pyridoxine dioctanoate and pyridoxine tripalmitate, vitamin B₁₂ and its derivatives, and vitamin B₁₅ and its derivatives; vitamin C, such as L-ascorbic acid, L-ascorbic acid dipalmitic ester, sodium (L-ascorbic acid)-2-sulfate and dipotassium L-ascorbic acid diphosphate; vitamin D, such as ergocalciferol and cholecalciferol; vitamin E, such as alpha-tocopherol, beta-tocopherol, gamma-tocopherol, dl-alpha-tocopheryl acetate, dl-alpha-tocopheryl nicotinate and dl-alpha-tocopheryl succinate. When present, in accordance with certain of the preferred embodiments, one or more vitamins may be included in effective amounts, advantageously from 0.0001-1% wt., preferably from 0.001-0.75% wt. based on the total weight of the liquid compositions of which it forms a part.

The inventive liquid compositions may include one or more chelating agents. Exemplary useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used, e.g. tetrasodium ethylenediaminetetraacetic acid. Such chelating agents may be omitted, or they may be included in generally minor amounts such as from 0.001-0.5% wt. based on the weight of the chelating agents and/or salt forms thereof. Desirably, when present, such chelating agents are included in the present inventive liquid compositions in amounts from 0.01-0.5% wt., preferably from about 0.01-0.2% wt.

The liquid compositions may optionally, but in some instances preferably, include a thickener constituent. Such include e.g., thickeners based on cellulose or one or more cellulose derivatives, and in certain preferred embodiments such a thickener constituent is necessarily present. Such are per se, known to the art and exemplary useful cellulose derivatives useful as a thickener constituent include methyl cellulose ethyl cellulose, hydroxymethyl cellulose hydroxy ethyl cellulose, hydroxy propyl cellulose, carboxy methyl cellulose, carboxy methyl hydroxyethyl cellulose, hydroxypropyl cellulose, hydroxy propyl methyl cellulose, ethylhydroxyethyl cellulose and ethyl hydroxy ethyl cellulose. Further useful thickeners may be one or more materials based on crosslinked polycarboxylate and/or polyacrylate polymer thickeners; including those typically exhibit a molecular weight from about 500,000 to about 4,000,000, and generally have degrees of crosslinking of from about 0.25% to about 15%. Such crosslinked polycarboxylate and/or polyacrylate polymers may include in their structure other monomers besides acrylic acid such as ethylene and propylene which act as diluents, and maleic anhydride which acts as a source of additional carboxylic groups. Such thickener constituents based on crosslinked polycarboxylate and/or polyacrylate polymer thickeners are widely commercially available and include, e.g., polycarboxylate polymers and/or polyacrylate polymers sold under trade names Carbopol®, Acrysol® ICS-1 and Sokalan®. Further examples of optional thickeners which may be are clay thickeners. Exemplary clay thickeners comprise, for example, colloid-forming clays, for example, such as smectite and attapulgite types of clay thickeners. The clay thickeners can be described as expandable layered clays, i.e., aluminosilicates and magnesium silicates. The term “expandable” as used to describe the instant clays relates to the ability of the layered clay structure to be swollen, or expanded, on contact with water. The expandable clays used herein are those materials classified geologically as smectites (or montmorillonite) and attapulgites (or polygorskites). Still further optional thickeners include one or more materials based on naturally occurring polysaccharide polymers such as xanthan gum, guar gum, locust bean gum, tragacanth gum, or derivatives thereof.

Yet further optional thickeners which may be advantageously used include one or more oxyalkylenated compound(s) having a molecular weight of at least about 2000, and which may comprise ethylene oxide groups (oxyethylenated compounds), propylene oxide groups (oxypropylenated compounds) or both (oxyethylenated/oxypropylenated compounds). Suitable oxyalkylenated compounds include, in particular, polyethylene glycols, polyethylene glycol esters and/or polypropylene glycol esters, polyethylene glycol ethers and/or polypropylene glycol ethers, alkoxylated aryl derivatives and in particular ethoxylated aryl polyol derivatives, oxyalkylenated and in particular oxyethylenated triesters of glycerol and of fatty acids, ethoxyethylenated urethane derivatives modified with alkyl chains, and mixtures thereof.

Exemplary polyethylene glycols which may be used in the composition of the invention include ethylene oxide polycondensates having a number of ethylene oxide (EO) units of greater than 10. The ethylene oxide number may range, for example, from 10 to 50 000 and preferably from 14 to 10 000. Suitable examples of polyethylene glycols include polyethylene glycol comprising 7 000 EO (CTFA name: PEG-7M), polyethylene glycol comprising 75 EO (CTFA name: PEG-75), polyethylene glycol comprising 20,000 EO (CTFA name: PEG-20M) and polyethylene glycol comprising 150 EO (CTFA name: PEG-150).

Also useful as oxyalkylenated compounds are polyethylene glycol esters and/or polypropylene glycol esters which are condensates of polyethylene glycol and/or polypropylene glycol with one or more fatty acids. These compounds have the formula:

RCOO-(EO)ₘ—(PO)ₙ—R′

in which m has a value of 0 to 300, n has a value of 0 to 300 the sum of m and n is 6 or greater, preferably 20 or greater, and R and R′ represent, independently of each other, hydrogen or a saturated or unsaturated, linear or branched, hydroxylated or non-hydroxylated alkyl chain containing from 1 to 200 carbon atoms, or an aryl chain, with the proviso that R and R′ are not simultaneously hydrogen.

Suitable, non-limiting examples of polyethylene glycol acid esters and/or polypropylene glycol acid esters include polyethylene glycol distearate (150 EO), the copolymer of polyethylene glycol ligand (30 EO) and of 12-hydroxystearic acid and polyethylene glycol stearate (40 EO), as well as such compounds based on polyoxyethylene/polyoxypropylene copolymers.

Further useful compounds include polyethylene glycol ethers and/or polypropylene glycol ethers are condensates of polyethylene glycol and/or polypropylene glycol with one or more fatty alcohols. These are compounds of formula:

R-(EO)ₘ—(PO)ₙ—R′

in which m has a value of 0 to 300, n has a value of 0 to 300 the sum of m and n is 6 or greater, preferably 20 or greater, and R and R′ represent, independently of each other, hydrogen or a saturated or unsaturated, linear or branched, hydroxylated or non-hydroxylated alkyl chain containing from 1 to 30 carbon atoms and preferably from 12 to 22 carbon atoms, or an aryl chain, with the proviso that R and R′ are not simultaneously hydrogen. Suitable, albeit non-limiting examples of such polyethylene glycol ethers include, oxyethylenated (30 EO) cetyl alcohol, oxyethylenated (30 EO) behenyl alcohol, oxyethylenated (23 EO) lauryl alcohol, oxyethylenated (20 EO) isocetyl alcohol, and, oxyethylenated (100 EO) stearyl alcohol. Suitable non-limiting examples of polyethylene glycol/polypropylene glycol ethers include oxyethylenated (5 EO) oxypropylenated (5 PO) lauryl alcohol, and oxyethylenated (25 EO) oxypropylenated (25 PO) lauryl alcohol. Further suitable oxyalkylenated compounds include the ethoxylated alkyl or aryl derivatives of polyol include, for example, oxyethylenated derivatives of fatty acid esters or of fatty alcohol ethers and of a polyol such as glycerol, sorbitol, glucose or pentaerythritol. Non-limiting examples of such compounds include, oxyethylenated (78 EO) glyceryl cocoate, oxyethylenated (120 EO) methylglucose dioleate, oxyethylenated (10 EO) polyglyceryl (2 mol of glycerol) laurate, and oxyethylenated (150 EO) pentaerythrityl tetrastearate. Still further suitable compounds include oxyalkylenated glyceryl triesters of fatty acids, for example, oxyethylenated (6 EO) caprylic/capric acid glycerides, and oxyethylenated (50 EO) olive oil.

A preferred oxyalkylenated compound of the topical compositions are polyethylene glycol diesters such as polyethylene glycol distearates ethoxylated with 50-350 units ethylene oxide. A particularly preferred material is Stepan PEG 6000 DS, described to be a polyethylene glycol distearate ethoxylated with 150 units of ethylene oxide.

Other oxyalkylenated compounds falling within the above descriptions, although not specifically disclosed herein but known to the art may also be used. Preferred oxyalkylenated compounds are disclosed with reference to one of more of the following Examples. The oxyalkylenated compounds may be present as single compounds or as mixtures of two or more oxyalkylenated compounds.

The oxyalkylenated compound(s) may be present in any effective amount.

Any of these thickener constituents, whether present singly or in combination with at least one further thickener constituent, may be present in any amount which is found effective in achieving a desired degree of thickening. When present, advantageously such thickener constituents may be present in amounts of from about 0.001% wt. to about 7.5% wt., preferably from about 0.01% wt. to about 5% wt., based on the total weight of the liquid composition of which it forms a part.

The compositions of the invention optionally include an ion source, preferably one or more inorganic salts. Such a source of ions may facilitate the thickening of the liquid compositions. It has been observed by the inventor that the presence of available ions favorably improve the thickening of the compositions, but at the same time do not undesirably detract from the storage stability of the liquid compositions. These ions may be added to the liquid compositions separately as a separate constituent, or they may be delivered as a part of a different material which is used in the liquid compositions and thereby become included in the same. Exemplary useful inorganic salts are water-soluble salts, including alkali metal salts, alkali hydroxides, alkali carbonates and alkali sulfates. Exemplary useful alkali metal salts include monovalent and divalent salts, such as one or more of: magnesium sulfate, calcium sulfate, potassium sulfate and sodium chloride.

When present, the ion source, especially wherein the ion source is an alkali metal salt, may be present in the liquid compositions in any effective amount, but advantageously are present in an amount of between 0.01% wt. and 5% wt., preferably is present in an amount of from 0.1% wt. to 2% wt., and still more preferably is present in an amount of between about 0.05% wt. and 0.5% wt., based on the total weight of the liquid compositions of which they form a part.

Optionally, one or more abrasive materials may also be included in the liquid compositions. By way of non-limiting examples such include one or more of: oxides, e.g., calcined aluminum oxides and the like, carbonates, e.g., calcium carbonate and the like, quartzes, siliceous chalk, diatomaceous earth, colloidal silicon dioxide, alkali metasilicates, e.g., sodium metasilicate and the like, perlite, pumice, feldspar, calcium phosphate, organic abrasive materials based on comminuted or particulate polymers especially one or more of polyolefins, polyethylenes, polypropylenes, polyesters, polystyrenes, acetonitrile-butadiene-styrene resins, melamines, polycarbonates, phenolic resins, epoxies and polyurethanes, natural materials such as, for example, rice hulls, corn cobs, and the like, or talc and mixtures thereof. The inclusion of an abrasive material may improve the removal of stains and dirt from dermal surfaces while the liquid composition is being applied thereto.

Separately from the at least one quaternary ammonium surfactant compound which is an essential constituent of the invention, the liquid compositions may optionally further include an ancillary antimicrobial agent. Examples of such ancillary antimicrobial agents one or more of: pyrithiones such as zinc pyrithione, halohydantoins such as dimethyldimethylol hydantoin, methylchloroisothiazolinone/methylisothiazolinone sodium sulfite, sodium bisulfite, imidazolidinyl urea, diazolidinyl urea, benzyl alcohol, 2-bromo-2-nitropropane-1,3-diol, formalin (formaldehyde), iodopropenyl butylcarbamate, chloroacetamide, methanamine, methyldibromonitrile glutaronitrile, glutaraldehyde, 5-bromo-5-nitro-1,3-dioxane, phenethyl alcohol, o-phenylphenol/sodium o-phenylphenol, sodium hydroxymethylglycinate, polymethoxy bicyclic oxazolidine, dimethoxane, thimersal dichlorobenzyl alcohol, captan, chlorphenenesin, dichlorophene, chlorbutanol, glyceryl laurate, halogenated diphenyl ethers such as 2,4,4-trichloro-2-hydroxy-diphenyl ether (Triclosan®) and 2,2-dihydroxy-5,5-dibromo-diphenyl ether, phenolic antimicrobial compounds such as mono- and poly-alkyl and aromatic halophenols, such as p-chlorophenol, methyl p-chlorophenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, and 5-chloro-2-hydroxydiphenylmethane, resorcinol and its derivatives, bisphenolic compounds such as 2,2-methylene bis(4-chlorophenol) and bis(2-hydroxy-5-chlorobenzyl)sulphide, benzoic esters (parabens), halogenated carbanilides such as 3-trifluoromethyl-4,4′-dichlorocarbanilide (Triclocarban), 3-trifluoromethyl-4,4-dichlorocarbanilide and 3,3,4-trichlorocarbanilide. The further antimicrobial agent may include one or more of: biguanides such as polyhexamethylene biguanide, p-chlorophenyl biguanide; 4-chlorobenzhydryl biguanide, 1,6-bis-(4-chlorobenzylbiguanido)-hexane (Fluorhexidine®), halogenated hexidine including, but not limited to, chlorhexidine (1,1′-hexamethylene-bis-5-(4-chlorophenyl biguanide) (Chlorohexidine®), as well as salts of any of the foregoing, e.g. polyhexamethylene biguanide hydrochloride.

Desirably, when present, such ancillary antimicrobial agents may be included in the inventive liquid compositions in any effective amount. Advantageously such amounts are from about 0.0001-2% wt., but preferably are from about 0.01-1% wt. of the liquid composition of which they form a part.

In certain particularly preferred embodiments, the inventive liquid compositions expressly exclude such ancillary antimicrobial agents as elucidated above.

The inventive liquid compositions may also comprise a peroxygen compound which may be essentially any compound containing a dioxygen (O—O) bond. Dioxygen bonds, particularly bivalent O—O bonds, are readily cleavable, thereby allowing compounds containing them to act as powerful oxidizers. Non-limiting examples of classes of peroxygen compounds include peracids, peracid salts, and peroxides such as hydrogen peroxide. The peroxygen can be any aliphatic or aromatic peracid (or peroxyacid) however, while any functional peroxyacid can be used, peroxyacids containing from 1 to 7 carbons are the most practical for use. These peroxyacids can include, but not be limited to, peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, and/or peroxybenzoic acid. Exemplary peracid salts include permanganates, perborates, perchlorates, peracetates, percarbonates, persulphates, and the like. Exemplary peroxide compounds include hydrogen peroxide, metal peroxides and peroxyhydrates. The metal peroxides that can be used include, but are not limited to, sodium peroxide, magnesium peroxide, calcium peroxide, barium peroxide, and/or strontium peroxide. Other salts (for example sodium percarbonate) have hydrogen peroxide associated therewith are also considered to be a source of hydrogen peroxide, thereby producing hydrogen peroxide in situ. Hydrogen peroxide is particularly preferred for use in the liquid compositions of the invention.

Desirably, when present, such peroxygen compounds may be advantageously included in amounts which are from about 0.1-10% wt., but preferably are from about 0.5-5% wt. of the liquid composition of which they form a part.

The liquid compositions of the invention may also include an acid constituent which may be used to adjust, and optionally buffer the pH of the liquid compositions to a desired pH value or range of pH values. Such may be one or more acids, including one or more organic acids and/or one or more inorganic acids. Exemplary inorganic acids, include sulfuric acid, phosphoric acid, potassium dihydrogenphosphate, sodium dihydrogenphosphate, sodium sulfite, potassium sulfite, sodium pyrosulfite (sodium metabisulfite), potassium pyrosulfite (potassium metabisulfite), acid sodium hexametaphosphate, acid potassium hexametaphosphate, acid sodium pyrophosphate, acid potassium pyrophosphate, hydrochloric acid, and sulfamic acid. Exemplary organic acids include those which generally include at least one carbon atom, and include at least one carboxyl group (—COOH) in its structure. Derivatives of said organic acids are also contemplated to be useful. Exemplary organic acid include linear aliphatic acids such as acetic acid; dicarboxylic acids, acidic amino acids, and hydroxy acids such as glycolic acid, lactic acid, hydroxyacrylic acid, alpha-hydroxybutyric acid, glyceric acid, malic acid, tartaric acid and citric acid, as well as acid salts of these organic acids. Particularly preferred acid constituents are disclosed in one or more of the Examples. When included, the acid constituent may be present 1 any effective amount, but is advantageously present in amount effective to establish the pH of the liquid compositions in the preferred range of between about 3.5 and about 7.5. Advantageously the acid constituent is present in an amount of from about 0.001-1% wt., preferably about 0.01-0.2% wt., yet more preferably between about 0.01-0.1% wt. when present in the liquid compositions.

In a further aspect, the present invention also contemplates a method for providing a germicidal benefit to skin or other topical surface which method contemplates the topical application of the inventive liquid compositions as described herein in a cleaning and/or germicidally effective amount. Preferably according to the foregoing method, a germicidal benefit is provided to the skin or other topical surface to which the composition has been applied. Preferred embodiments of the topical germicidal compositions exhibit good germicidal efficacy of undesired microorganisms, especially: E. coli, P. aeruginosa, and Vanomycin resistant Enterococcus on dermal (viz., skin. body) surfaces. Advantageously the inventive liquid compositions exhibit further antimicrobial efficacy against one or more of certain gram positive pathogens, certain gram negative pathogens, certain viruses, certain fungi and/or certain molds as well.

The inventor has surprisingly discovered that notwithstanding the long standing prejudices in the art cautioning against the use of combinations of anionic surfactants with one or more germicidally effective cationic surfactants in largely aqueous compositions as an expected complexing reaction occurs between these different chemical species causing the deactivation of the cationic surfactants and elimination of their germicidal benefit, such a prejudice is overcome by the liquid compositions in the present invention. While not wishing to be bound by the following, it is believed that inclusion of both the germicidally effective cationic surfactant compound in conjunction with the cationic conditioner constituent in the specified ratios, and further the use of the specified types of anionic surfactants disclosed herein, also within specific ratios, unexpectedly provides for a system wherein the germicidal efficacy of the liquid composition is not unduly diminished, as evidenced by the excellent germicidal benefits provided. While it believed that the germicidally effective cationic surfactant compound does form a complex with the anionic surfactant compounds present to form an anionic-cationic complex, this complex can disassociate to release cations at a biologically effective concentration when encountering a microorganism, which causes an abstraction of the cation from the anionic-cationic complex as the cation now associates with and interacts with the microorganism. This changes the equilibrium concentration of the liquid composition, due to the reduction of the cations in the liquid composition. The disassociated (or now “un-complexed”) anionic surfactant compound forms a new anionic-cationic complex with either a further molecule of the germicidally effective cationic surfactant or with a molecule of the cationic conditioner constituent, which as before, complex can disassociate to release cations at a biologically effective concentration when encountering a microorganism, which causes an abstraction of the cation from the anionic-cationic complex as the cation associates and interacts with the microorganism, again providing a disassociated anionic surfactant compound, which can then form a new complex with a further available further molecule of the germicidally effective cationic surfactant or with a molecule of the cationic conditioner constituent. In such a manner, a supply of cations may be supplied by the liquid composition to microorganisms, particularly undesired microorganisms such as gram positive and gram negative microorganisms, which cations reduce the viability of such microorganisms with which they come into contact or become associated with.

It is to be further expressly understood that the liquid compositions disclosed herein may be topically applied to the skin on any part of the body, e.g., the skin on the face, neck, chest, back, arms, axilla, hands, legs, and scalp. The liquid compositions disclosed herein may also be used on the hair. It is contemplated that in use, the consumer dispenses a quantity of the liquid composition described herein and applies it to the skin or any other part of the body where they may be retained upon but is beneficially rubbed into the applied skin or other part of the body by the consumer to provide both a germicidal benefit and/or cleaning benefit to the treated skin or other part of the body. Advantageously the thus applied liquid composition is allowed to remain on the skin or other part of the body to which it has been applied, without any subsequent washing or rinsing. However, if desired by a consumer, the liquid compositions may be rinsed by the consumer under a stream of running water, e.g, in a shower or by immersion into water, e.g, a bath. Thus, a further aspect of the invention is directed to the use of the liquid compositions as described herein as a dermal soap-type product used for cleaning and treating body surfaces.

The following examples below illustrate exemplary formulations as well as preferred embodiments of the invention. It is to be understood that these examples are provided by way of illustration only and that further useful formulations falling within the scope of the present invention and the claims may be readily produced by one skilled in the art without deviating from the scope and spirit of the invention.

### EXAMPLES

In the following compositions, the constituents were used “as supplied” from their respective suppliers

Examples the inventive liquid compositions are described in the following Table 1; the constituents indicated on Table 1 used to produce the formulations were used on an “as supplied” basis, and may constitute less than 100% wt. “actives”, or may have been supplied as constituting 100% wt. “active” of the named compound, as indicated on Table 1. The amounts indicated on Table 1 refer to % wt. of the named constituent used in a composition, based on a total weight of 100% wt. for an identified composition. The identity of these constituents are disclosed in more detail on Table 2. The liquid compositions were produced by mixing the constituents into water as outlined in Table 1 in a beaker at room temperature which was stirred with a conventional magnetic stirring rod or paddle mixer; stirring continued until the formulation was homogenous in appearance. It is to be noted that the constituents might be added in any order, but it is preferred that a first premixture is made of any fragrance constituent with one or more surfactants in an aliquot of water used in the inventive liquid compositions. The order of addition is not critical, but good results are obtained where the surfactants (which may be also the premixture of the fragrance and surfactants) are produced prior to the addition of the remaining constituents to the water.

TABLE 1

| | E1 | E2 | E3 | E4 | E5 | E6 | E7 | E8 |
|---|---|---|---|---|---|---|---|---|
| thickener1 | — | — | 0.1 | 0.1 | — | — | — | — |
| thickener2 | 1.25 | 0.8 | 0.8 | 0.8 | 0.8 | 0.8 | 0.8 | 1.0 |
| quaternary | 0.26 | 0.26 | 0.26 | 0.26 | 0.26 | 0.26 | 0.26 | — |
| ammonium1 | | | | | | | | |
| quaternary | — | — | — | — | — | — | — | 0.13 |
| ammonium2 | | | | | | | | |
| sarcosinate | 10.0 | 10.0 | 10.0 | 10.0 | 10.0 | 3.0 | 3.0 | 3.0 |
| surfactant | | | | | | | | |
| betaine | 6.0 | 5.0 | 5.0 | 5.0 | 5.0 | 5.0 | 5.0 | 5.0 |
| surfacatant | | | | | | | | |
| (30%) | | | | | | | | |
| amine oxide | 3.0 | 3.0 | 3.0 | 3.0 | 3.0 | 2.0 | 2.0 | 2.0 |
| cetrimonium | 6.5 | 6.5 | 6.5 | 6.5 | 8.0 | 12.0 | 12.0 | 12.0 |
| chioride1 | | | | | | | | |
| cetrimonium | — | — | — | — | — | — | — | — |
| chloride2 | | | | | | | | |
| laureth-13 | — | — | 5.0 | 5.0 | 5.0 | 2.0 | 2.0 | 2.0 |
| carboxylate | | | | | | | | |
| amphoteric | — | — | — | — | — | — | — | — |
| surfactant | | | | | | | | |
| EDTA | 0.25 | 0.25 | 0.25 | 0.25 | 0.25 | 0.25 | 0.25 | 0.25 |
| LDEA | — | — | — | — | — | — | 0.57 | — |
| Kathon CG | 0.02 | 0.02 | 0.02 | 0.02 | 0.02 | 0.02 | 0.02 | 0.02 |
| hydrogen | — | — | — | — | — | — | — | — |
| peroxide (50%) | | | | | | | | |
| sodium | — | — | — | 1.5 | — | 0.92 | 0.9 | 0.4 |
| chloride (20%) | | | | | | | | |
| citric acid | 0.26 | 0.28 | 0.26 | 0.26 | 0.29 | 0.55 | 0.72 | 0.58 |
| (50%) | | | | | | | | |
| pH | 6.59 | 6.53 | 6.48 | 6.48 | 6.32 | 4.91 | 4.87 | 4.90 |

| | E9 | E10 | E11 | E12 | E13 | E14 |
|---|---|---|---|---|---|---|
| thickener1 | — | — | — | — | — | — |
| thickener2 | 1.0 | 1.0 | 0.8 | 0.8 | 0.8 | 0.8 |
| quaternary ammonium1 | — | — | — | — | — | — |
| quaternary ammonium2 | 0.13 | 0.13 | 0.104 | 0.14 | 0.14 | 0.14 |
| sarcosinate surfactant | 3.0 | 3.0 | 2.4 | 2.43 | 3.0 | 2.43 |
| betaine surfacatant (30%) | 5.0 | 5.0 | 4.0 | 4.05 | 4.0 | 2.6 |
| amine oxide | 2.0 | 2.0 | 1.6 | 1.62 | 1.6 | 1.62 |
| cetrimonium chloride1 | 12.0 | — | — | — | 12.0 | — |
| cetrimonium chloride2 | — | 10.0 | 10.1 | 10.1 | — | 10.1 |
| laureth-13 carboxylate | 2.0 | 1.0 | 0.8 | 0.81 | 2.0 | 0.81 |
| amphoteric surfactant | — | — | — | — | — | 1.0 |
| EDTA | 0.25 | 0.25 | 0.2 | 0.45 | 0.25 | — |
| LDEA | 0.57 | 0.57 | 0.74 | 0.79 | 0.74 | 0.79 |
| preservative | 0.02 | 0.02 | 0.02 | 0.02 | 0.02 | — |
| hydrogen peroxide (50%) | — | — | — | — | — | 5.0 |
| sodium chloride (20%) | 0.4 | — | — | — | — | — |
| citric acid (50%) | 0.58 | 0.59 | 0.74 | 0.79 | 0.74 | 0.79 |
| pH | 4.96 | 4.77 | 4.62 | 4.49 | 4.9 | 3.99 |
| hydrogen peroxide stability | — | — | — | — | — | 93.5% |

The identity of the individual constituents used to produce the liquid compositions of Table 1 are described more fully on Table 2.

TABLE 2

| | |
|---|---|
| thickener1 | hydroxypropyl methyl cellulose (100% wt. |
| | actives), supplied as Methocel 865N (ex. |
| | DOW Corp.) |
| thickener2 | ethoxylated polyethylene glycol distearate. |
| | (100% wt. actives), supplied as PEG 6000 DS |
| | (ex. The Hallstar Company) |
| quaternary ammonium1 | alkyl dimethyl benzyl ammonium chlorides |
| | (50% wt. actives), supplied as Barquat MB50 |
| | (ex. Lonza) |
| quaternary ammonium2 | benzethonium chloride (100% wt. actives), |
| | supplied as Hyamine 1622 Crystal (ex. |
| | Lonza) |
| sarcosinate surfactant | sodium lauroyl sarcosinate, (30% wt. actives) |
| | supplied as Crodasinic LS-30 (ex. Croda Inc.) |
| betaine surfacatant (30%) | cocoamidopropyl dimethyl betaine, (30% wt. |
| | actives) supplied as Empigen BS/FA (ex. |
| | Cognis) |
| amine oxide | lauryl dimethyl amine oxide (30% wt. actives), |
| | supplied as Ammonyx LO (ex. Stepan Co) |
| cetrimonium chloride1 | cetrimonium chloride, (25% wt. actives), |
| | supplied as Ammonyx CETAC 25 (ex. Stepan |
| | Co.) |
| cetrimonium chloride2 | cetrimonium chloride, (30% wt. actives), |
| | supplied as Ammonyx CETAC 30 (ex. Stepan |
| | Co.) |
| laureth-13 carboxylate | sodium laureth-13 carboxylate, (79% wt. |
| | actives) supplied as Miranate LEC80 (ex. |
| | Rhodia) |
| amphoteric surfactant | amphoteric surfactant, (30% wt. actives), |
| | supplied as Tomamine Amphoteric SC (ex. |
| | Air Products Inc.) |
| EDTA | tetrasodium ethylene diamine tetraacetic acid |
| | (38.9% wt. actives), supplied as Trilon BX |
| | (ex. BASF) |
| LDEA | lauryl diethanolamide (100% wt. actives), |
| | supplied as Ninol 55L (ex. Stepan Co.) |
| preservative | preservative, supplied as Kathon CG |
| hydrogen peroxide (50%) | aqueous hydrogen peroxide composition; |
| | (50% available oxygen) |
| sodium chloride (20%) | aqueous dilution of NaCl, 20% wt. |
| | concentration |
| citric acid (50%) | aqueous dilution of citric acid, 50% wt. |
| | concentration |

Several of the foregoing compositions were tested and evaluated according to one or more of the following test protocols.

### Evaluation of Germicidal Efficacy

Several formulations according to the invention as described from Table 1 were also evaluated in order to evaluate their antimicrobial efficacy against Staphylococcus aureus (ATCC 6538), Escherichia coli (ATCC 10536), Pseudomonas aeruginosa (ATCC 15442) and a strain of Vancomycin resistant Enteroccosu faecalis (ATCC 51299) in accordance with the protocols of British Standard EN 1276, a quantitative suspension test for the evaluation of bactericidal activity of chemical disinfectants and antiseptics used in food, industrial, domestic and institutional environments—Test Method and requirements (phase 2, step 1) Ref. No. EN 1276: 1997E the contents of which are herein incorporated by reference. The protocols of this “dirty” test were followed with the following variations from the published protocol: the tested formulations of Table 1 were diluted in sterile deionized water at 90% v/v or 80% v/v (composition to water ratio) and were tested for a either a 1 minute or 5 contact time; a final concentration of 3 g/l bovine albumin was used in testing for dirty conditions (Section 5.2.2.8.2, part b). A specified in the protocol of British Standard EN 1276 a reduction in viability was calculated for each test system for each of the microorganisms. The results of this test per the British Standard EN 1276 is reported on the following Table 3.

TABLE 3

| | Log₁₀ reduction | | | | |
|---|---|---|---|---|---|
| | | | | | Vanomycin |
| | dilution in | | | | resistant |
| | water | S. aureus | P. aeruginosa | E. coli | Enterococcus |
| E7 | 90% | >5.18 | 4.54 | — | — |
| E8 | 90% | >5.18 | >5.63 | 2.54 | >5.34 |
| E12 | 80% | >5.18 | >5.63 | 5.05 | >5.34 |

A further formulation as described on Table 1 was similarly evaluated to the compositions tested with reference to Table 3. This further formulation (E12) was tested using a sample which was prepared and thereafter stored at room temperature for at least 6 months prior to the testing, prior to testing the E12 formulation was diluted in sterile deionized water at 50% v/v or 80% v/v (composition to water ratio) and these dilutions were tested for a 1 minute contact time. The results of the testing is reported on following Table 4.

TABLE 4

| | Log₁₀ reduction | | | | |
|---|---|---|---|---|---|
| | | | | | Vanomycin |
| | dilution in | | | | resistant |
| | water | S. aureus | P. aeruginosa | E. coli | Enterococcus |
| E12 | 80% | 4.06 | >6.46 | 5.87 | >6.46 |
| E12 | 50% | 5.15 | >6.46 | >6.47 | >6.46 |

As may be seen from the results indicated above, the tested compositions exhibited excellent germicidal benefits, and in most cases exhibited in excess of a 4 log₁₀ reduction of each of the four test microorganisms, as reported on Table 3, including Vanomycin resistant Enterococcus.

### Storage Stability:

All of the compositions of Table 1 were tested for their storage stability under the following test storage conditions: (A) at 40° C. for 4 months, (B) at 50° C. for 4 weeks; (C) at 60° C. for 2 weeks, and (D) at 40° C. for 6 months. Periodically during as well as following the conclusion of the test, all of the compositions of Table 1 did not suffer phase separation, precipitation or more than a 5% change from their initial pH prior to being subjected to the above test storage conditions.

While the invention is susceptible of various modifications and alternative forms, it is to be understood that specific embodiments thereof have been shown by way of example in the drawings which are not intended to limit the invention to the particular forms disclosed; on the contrary the intention is to cover all modifications, equivalents and alternatives falling within the scope and spirit of the invention as expressed in the appended claims.

1. An aqueous liquid composition for application to the epidermis which provides a germicidal benefit thereto comprising:
0.02-10% wt. of a germicidally effective amount of a quaternary ammonium surfactant compound, which simultaneously functions as a source of cations and also to impart a germicidal benefit to the epidermis upon which the liquid composition is applied;
0.1-25% wt. of at least one anionic surfactant constituent which excludes anionic surfactants of the sulfate and sulfonate types, which one or more of the anionic surfactant constituents simultaneously provide a cleaning benefit and also function as a source of anions;
0.1-15% wt. of a cationic conditioner constituent, which simultaneously functions as an additional source of cations;
0-10% wt. of a foam stabilizer constituent; and
at least 70% wt. of water.

2. The composition according to claim 1, wherein the quaternary ammonium surfactant compound comprises the structural formula: 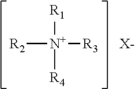
wherein at least one of R₁, R₂, R₃ and R₄ comprises an alkyl, aryl or alkylaryl substituent of from 6 to 26 carbon atoms.

3. The composition according to claim 2, wherein, R₂ and R₃ comprise C₈-C₁₂alkyl.

4. The composition according to claim 1, wherein at least one of the anionic surfactant constituents comprises an alkyl ether carboxylate.

5. The composition according to claim 1, wherein at least one of the anionic surfactant constituents comprises a sarcosinate surfactant.

6. The composition according to claim 1, wherein the weight ratio of the germicidally effective amount of quaternary ammonium surfactant compound and the cationic conditioner constituent to the at least one anionic surfactant constituent is not less than 0.6.

7. The composition according to claim 1, wherein the weight ratio of the germicidally effective amount of quaternary ammonium surfactant compound to the cationic conditioner constituent is less than 0.14.

8. The composition according to claim 1, wherein the weight ratio of the cationic conditioner constituent to the at least one anionic surfactant constituent is at least 0.5.

9.-10. (canceled)

11. The composition according to claim 1, wherein the pH of the liquid composition is between about 3.5 and about 7.5.

12. The composition according to claim 1 further comprising a constituent that imparts an aesthetic benefit to the composition.

13. The composition according to claim 12, wherein the aesthetic benefit to the composition is selected from the group consisting of color and fragrance.

14. The composition according to claim 1 further comprising a constituent that imparts a technical benefit to the composition.

15. The composition according to claim 14, wherein the technical benefit comprises viscosity adjustment.

16. The composition according to claim 2, wherein at least one of R₁, R₂, R₃ and R₄ comprises one or more amide, ether or ester linkages.

17. The composition according to claim 2, wherein the entire cation portion of the molecule has a molecular weight of at least 165.

18. The composition according to claim 2, wherein the counterion X comprises a salt-forming anion that permits water solubility or water miscibility of the quaternary ammonium complex.

19. The composition according to claim 2, wherein R₂ is selected from the group consisting of C₁₂₋₁₆alkyl, C₈₋₁₈alkylethoxy, and C₈₋₁₈alkylphenolethoxy.

20. The composition according to claim 2, wherein R₃ comprises benzyl.

21. The composition according to claim 2, wherein X is selected from the group consisting of a halide and a methosulfate anion.

22. The composition according to claim 1, wherein the weight ratio of the germicidally effective amount of quaternary ammonium surfactant compound and the cationic conditioner constituent to the at least one anionic surfactant constituent is at least 1.

23. The composition according to claim 1, wherein the weight ratio of the germicidally effective amount of quaternary ammonium surfactant compound and the cationic conditioner constituent to the at least one anionic surfactant constituent is at least 1.5.

24. The composition according to claim 1, wherein the weight ratio of the germicidally effective amount of quaternary ammonium surfactant compound and the cationic conditioner constituent to the at least one anionic surfactant constituent is at least 3.0.

25. The composition according to claim 1, wherein the weight ratio of the cationic conditioner constituent to the at least one anionic surfactant constituent is at least 1.

26. The composition according to claim 1, wherein the weight ratio of the cationic conditioner constituent to the at least one anionic surfactant constituent is at least 2.

27. The composition according to claim 1, wherein the composition is effective against a microorganism selected from the group consisting of E. coli, P. aeruginosa, and Vancomycin resistant Enterococcus faecalis (“VRE”).

28. An aqueous liquid composition for application to the epidermis which provides a germicidal benefit thereto comprising:
0.02-10% wt. of a germicidally effective amount of a quaternary ammonium surfactant compound, which simultaneously functions as a source of cations and also to impart a germicidal benefit to the epidermis upon which the liquid composition is applied;
0.1-25% wt. of at least one anionic surfactant constituent that excludes anionic surfactants of the sulfate and sulfonate types, which one or more of the anionic surfactant constituents simultaneously provide a cleaning benefit and also function as a source of anions;
0.1-15% wt. of a cationic conditioner constituent, which simultaneously functions as an additional source of cations;
0-10% wt. of a foam stabilizer constituent; and
at least 70% wt. of water;
wherein the weight ratio of the germicidally effective amount of quaternary ammonium surfactant compound and the cationic conditioner constituent to the at least one anionic surfactant constituent is not less than 0.6;
wherein the weight ratio of the germicidally effective amount of quaternary ammonium surfactant compound to the cationic conditioner constituent is less than 0.14;
wherein the weight ratio of the cationic conditioner constituent to the at least one anionic surfactant constituent is at least 0.5; and
wherein the pH of the liquid composition is between about 3.5 and about 7.5.

29. The composition according to claim 28, wherein the quaternary ammonium surfactant compound comprises the structural formula: 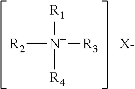
wherein at least one of R₁, R₂, R₃ and R₄ comprises an alkyl, aryl or alkylaryl substituent of from 6 to 26 carbon atoms that may be straight chained or branched and optionally comprises one or more amide, ether or ester linkages;
wherein the entire cation portion of the molecule has a molecular weight of at least 165; and
wherein the counterion X comprises a salt-forming anion that permits water solubility or water miscibility of the quaternary ammonium complex.

30. The composition according to claim 29, wherein R₂ and R₃ are the same or different C₈-C₁₂alkyl.

31. The composition according to claim 29, wherein R₂ is selected from the group consisting of C₁₂₋₁₆alkyl, C₈₋₁₈alkylethoxy, and C₈₋₁₈alkylphenolethoxy;
wherein R₃ comprises benzyl; and
wherein X is selected from the group consisting of a halide and a methosulfate anion.

32. The composition according to claim 28, wherein the composition is effective against a microorganism selected from the group consisting of E. coli, P. aeruginosa, and Vancomycin resistant Enterococcus faecalis (“VRE”).

33. An aqueous liquid composition for application to the epidermis which provides a germicidal benefit thereto comprising:
0.02-10% wt. of a germicidally effective amount of a quaternary ammonium surfactant compound, which simultaneously functions as a source of cations and also to impart a germicidal benefit to the epidermis upon which the liquid composition is applied;
0.1-25% wt. of at least one anionic surfactant constituent that excludes anionic surfactants of the sulfate and sulfonate types, at least one of the anionic surfactant constituents selected from the group consisting of an alkyl ether carboxylate and a sarcosinate surfactant, which one or more of the anionic surfactant constituents simultaneously provide a cleaning benefit and also function as a source of anions;
0.1-15% wt. of a cationic conditioner constituent, which simultaneously functions as an additional source of cations;
0-10% wt. of a foam stabilizer constituent; and
at least 70% wt. of water;
wherein the weight ratio of the germicidally effective amount of quaternary ammonium surfactant compound and the cationic conditioner constituent to the at least one anionic surfactant constituent is at least 3;
wherein the weight ratio of the germicidally effective amount of quaternary ammonium surfactant compound to the cationic conditioner constituent is less than 0.14;
wherein the weight ratio of the cationic conditioner constituent to the at least one anionic surfactant constituent is at least 2; and
wherein the pH of the liquid composition is between about 3.5 and about 7.5.

34. The composition according to claim 3, wherein the composition is effective against at least two microorganisms selected from the group consisting of E. coli, P. aeruginosa, and Vancomycin resistant Enterococcus faecalis (“VRE”).
